(19) United States
(12) Patent Application Publication
(10) Pub. No.: US 2024/0299120 A1
(43) Pub. Date: Sep. 12, 2024

(54) STABILIZING FRAMES AND METHODS OF POSITIONING WITH A STABILIZING FRAME

(71) Applicant: The Cleveland Clinic Foundation, Cleveland, OH (US)

(72) Inventors: Richard Rammo, Cleveland, OH (US); Stephen Harasimchuk, Parma, OH (US); Francesco Pucci, Chicago, IL (US); Sebastian Bakalarczyk, Parma, OH (US); William Bingaman, Kirtland, OH (US); Demitre Serletis, Moreland Hills, OH (US)

(21) Appl. No.: 18/599,646

(22) Filed: Mar. 8, 2024

Related U.S. Application Data
(60) Provisional application No. 63/450,869, filed on Mar. 8, 2023.

(51) Int. Cl. A61B 90/14

(52) U.S. Cl. CPC A61B 90/14

(57) ABSTRACT

Stabilizing frames comprise a base extending at least 180 degrees about an axis of the stabilizing frame to define a reception area. The stabilizing frames further comprise a plurality of anchors configured to be embedded within a skull of a patient. The stabilizing frames further comprise a registration fiducial mounted relative to the base, the registration fiducial comprising a recess configured to receive a distal tip of a registration probe of a robot. Methods of positioning a patient's head relative to a robot for a surgical procedure comprise positioning a patient's head and/or neck within a reception area of a stabilizing frame comprising a registration fiducial. The methods further comprise removably fixedly attaching the stabilizing frame to the skull and the stabilizing frame to the robot. The methods still further comprise inserting a tip of a probe of the robot into a recess of the registration fiducial.

### CROSS-REFERENCE TO RELATED APPLICATIONS

This application claims the benefit of U.S. Provisional Application No. 63/450,869, filed Mar. 8, 2023, which is hereby incorporated by reference in its entirety.

### FIELD

The present disclosure relates generally to stabilizing frames and methods of positioning with a stabilizing frame and, more particularly, to stabilizing frames including a registration fiducial and methods of positioning a patient's head relative to a robot for a surgical procedure with a stabilizing frame including a registration fiducial.

### BACKGROUND

Epileptic seizures in patients are often difficult to treat with medication alone. For difficult cases to treat, precise problem areas of the brain can be located using stereoelectroencephalography (SEEG). SEEG is a minimally invasive surgical procedure designed to locate areas in the brain where the seizures begin. During SEEG, neurosurgeons place electrodes through the skull to provide more accurate readings to pinpoint the precise locations where the epileptic seizures begin. Once located, subsequent techniques can be used to treat the precise locations to reduce or prevent further epileptic seizures from occurring.

Currently, in preparation for the SEEG, incisions are made in the scalp and fiducial anchors are mounted directly into the skull of the patient. Then a stabilizing frame is mounted to the head of the patient such as a LEKSELL® Coordinate Frame G stabilizing frame available from Elektra. The stabilizing frame can be used to fixedly mount the patient's head to an SEEG robot such as a ROSA ONE® brain application robot available from Zimmer Biomet. Once the patient's head is fixed relative to the robot with the stabilizing frame, a probe from the robot interfaces with each one of the plurality of fiducial anchors mounted to the skull so that the exact orientation of the imaging loaded into the software can be matched to the physical location of the patients brain relative to the robot. Once calibrated, the SEEG surgical procedure can proceed with very precise placement of the electrodes.

To further increase the accuracy, there is a need to increase the number and location of the fiducials. At the same time, there is a desire to reduce the number of incisions into the scalp and simplify the surgical procedure.

### SUMMARY

There are set forth herein benefits that can be achieved by mounting the fiducials relative to a base of a stabilizing frame rather than invasively anchoring the fiducials into the skull. Mounting the fiducials relative to the base as part of the stabilizing frame can provide a wide range of benefits. For instance, the positions of the fiducials can be adjustable to various positions relative to the base without injury to the scalp or skull. Furthermore, the fiducials can be positioned at locations other than near the topography of the skull. Still further, an increased number of fiducials can be used without requiring an increased number of incisions or invasive anchoring sites into the skull. The increased number of fiducials can be beneficial to increase the accuracy of image registration. Still further, the time necessary to place the fiducials is reduced since mounting the fiducials relative to the base of the stabilizing frame can take less time than surgically implanting the fiducials into the skull of the patient. In some embodiments, the registration between the fiducials and the robot can also be configured to enhance the overall surgical procedure.

In accordance with aspects of the disclosure, a stabilizing frame comprises a base extending at least 180 degrees about an axis of the stabilizing frame to define a reception area. The stabilizing frame further comprises a plurality of anchors mounted relative to the base, wherein the plurality of anchors are configured to be embedded within a skull of a patient. The stabilizing frame further comprises a registration fiducial mounted relative to the base, the registration fiducial comprising a recess configured to receive a distal tip of a registration probe of a robot.

In accordance with further aspects of the disclosure, a method of positioning a patient's head relative to a robot for a surgical procedure comprises positioning a patient's head and/or neck within a reception area of a stabilizing frame comprising a registration fiducial. The method further comprises anchoring the stabilizing frame to a skull of the head to removably fixedly attach the stabilizing frame to the skull. The method still further comprises removably fixedly attaching the stabilizing frame to the robot to prevent the head from moving relative to a body of the robot. The method still further comprises inserting a tip of a probe of the robot into a recess of the registration fiducial.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

The above and other features and advantages of embodiments of the present disclosure are better understood when the following detailed description is read with reference to the accompanying drawings, in which:

FIG. 1 is a perspective view of an example embodiment of a stabilizing frame in accordance with aspects of the disclosure;

FIG. 2 is a top view of an embodiment of a front bracket of the stabilizing frame of FIG. 1;

FIG. 3 is a front view of the front bracket of FIG. 2;

FIG. 4 is a top view of an embodiment of a rear bracket of the stabilizing frame of FIG. 1;

FIG. 5 is a front view of the rear bracket of FIG. 4;

FIG. 6 is a cross-sectional view of the registration fiducial along line 6-6 of FIG. 5;

FIG. 7 is an enlarged view a tip of a registration probe of a robot inserted within a recess of the registration fiducial of FIG. 6;

FIG. 8 is an enlarged view a tip of a registration probe of a robot inserted within a recess of another registration fiducial in accordance with another embodiment of the disclosure;

FIG. 9 is a cross-sectional view along line 9-9 of FIG. 8, illustrating the registration probe of the robot being inserted into the recess of the registration fiducial of FIG. 8;

FIG. 10 illustrates methods of positioning a patient's head relative to a robot for a surgical procedure; and

FIG. 11 illustrates inserting a tip of a probe into a recess of the registration fiducial.

### DETAILED DESCRIPTION

Embodiments of the present disclosure will now be described with reference to the drawings, wherein like reference numerals are used to refer to like elements throughout. It will be understood that the figures are not necessarily to scale.

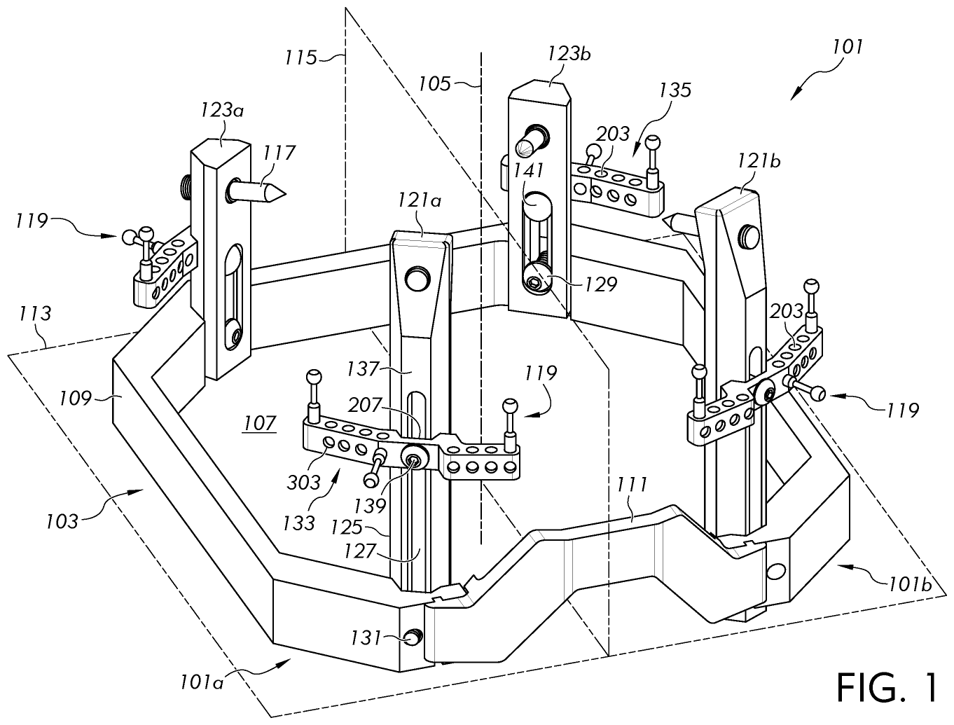

Referring to FIG. 1, the present disclosure can comprise a stabilizing frame 101 that can be used to stabilize the head of a patient and immobilize the head relative to a robot during a medical procedure. A wide range of stabilizing frame configurations can be used in accordance with aspects of the disclosure. For instance, in one example, a LEKSELL® Coordinate Frame G stabilizing frame available from Elektra can be used in accordance with aspects of the disclosure although other stabilizing frames can be provided in further embodiments. As shown, the stabilizing frame 101 can comprise a base 103 that can extend at least 180 degrees about an axis 105 of the stabilizing frame 101 to define a reception area 107. In some embodiments, as shown, the base 103 can extend a full 360 degrees about the axis 105 to define the reception area 107. Extending the base 103 the full 360 degrees about the axis 105 to define the reception area 107 can increase the strength and rigidity of the stabilizing frame 101 to resist bending when anchoring the stabilizing frame 101 to the skull of the patient. In some embodiments, the base 103 can comprise a one-piece circular hoop that can be formed of a rigid material such as metal. Although a single one-piece hoop can be provided, in further embodiments, the circular hoop may be fabricated from a plurality of segments fastened together. For instance, as shown in FIG. 1, the base 103 can comprise a C-shaped member 109 and a bridge 111 that can be attached to form the rigid assembled circumferential hoop extending the full 360 degrees. Although not shown, alternative base members may comprise a C-shaped member extending 180 degrees or more.

Although not required, in some embodiments, at least portions of the base can extend along a plane. For example, as shown, the C-shaped member 109 of the base 103 can extend along a base plane 113 and the axis 105 can extend perpendicular to the base plane 113. In some embodiments, as shown, the axis 105 can comprise a symmetrical axis extending along a symmetrical plane 115 that is perpendicular to the base plane 113. As shown, in some embodiments, the symmetrical plane 115 can bisect the stabilizing frame 101 into a right frame portion 101a and a left frame portion 101b that is a mirror image of the right frame portion 101a about the symmetrical plane 115.

The stabilizing frame 101 can further comprise a plurality of anchors 117 mounted relative to the base 103, wherein the plurality of anchors 117 are configured to be embedded within a skull of a patient. The stabilizing frame 101 can further comprise one or more registration fiducials 119 mounted relative to the base 103.

In some embodiments, the plurality of anchors 117 and/or the one or more registration fiducials 119 can be directly mounted to the base. For example, although not shown, the plurality of anchors 117 may be threadedly mounted within bores in the C-shaped member 109 of the base 103 and configured to be embedded within the skull of a patient to removably mount the stabilizing frame to the skull of the patient. Furthermore, the one or more registration fiducials 119 can be mounted directly to the base 103 wherein mounting of the base to the skull of the patient with the plurality of anchors likewise mounts the registration fiducials 119 relative to the skull of the patient by way of the base 103 of the stabilizing frame.

In alternative embodiments, as shown, the plurality of anchors 117 can be mounted relative to the base 103 indirectly by a plurality of arms 121a, 121b, 123a, 123b mounted to the base 103. For instance, the plurality of arms can comprise a pair of front arms 121a, 121b comprising a right front arm 121a and a left front arm 121b and a pair of rear arms 123a, 123b comprising a right rear arm 123a and a left rear arm 123b. While four arms are illustrated, two, three, or more than four arms may be provided in further embodiments. Each arm of the plurality of arms can comprise a segment 125 extending away from the base 103 in a direction of the axis 105. In some embodiments, the segment can extend at an angle relative to the axis while still extending generally in the direction of the axis 105. In alternative embodiments, the segment 125 can extend parallel to the axis 105. For instance, each arm of the plurality of arms 121a, 121b, 123a, 123b comprises a lower segment 125 that can be parallel to the axis 105 of the stabilizing frame 101.

As shown in FIG. 1, a corresponding anchor of the plurality of anchors 117 can be mounted to a distal end portion of each arm of the plurality of arms 121a, 121b, 123a, 123b. The anchors can each comprise a tip configured to embed within the skull of a patient. Furthermore, the anchors 117 can be designed with a predetermined angular approach to engage the skull at an optimum orientation to facilitate embedding of the tip of the anchor within the skull such as an angle expected to be substantially perpendicular to a plane of tangency at the point where the anchor is expected to engage the surface of the skull.

In some embodiments, one or more arms of the plurality of arms can comprise a slot extending in the direction of the axis 105. For instance, as shown in FIG. 1, each arm of the plurality of arms 121a, 121b, 123a, 123b can comprise an elongated slot 127 extending in the direction of the axis 105. A threaded shank 131 of a screw 129 can be threadingly received within a threaded aperture of the base 103 wherein the screw 129 can be tightened to lock the position of each arm relative to the base 103. As such, the position of the arm relative to the base 103 can be adjusted, for example, to adjust the position of each arm, and consequently the position of each anchor associated with each arm, relative to the base 103 in the direction of the axis 105.

In some embodiments, the registration fiducial 119 can be mounted relative to an arm of the stabilizing frame 101. For example, one or more registration fiducials 119 can be mounted relative to one or more of the plurality of arms 121a, 121b, 123a, 123b. Although one or more fiducials are shown mounted relative to all of the arms, one or more registration fiducials may not be mounted relative to one or more of the arms. Furthermore, although not shown, additional registration fiducials may be mounted relative to one or more of the arms or mounted directly to the base in addition to mounting relative to the one or more arms. In the illustrated embodiment, the right front arm 121a and the left front arm 121b each comprise a pair of registration fiducials 119 with the arm laterally positioned between the corresponding pair of registration fiducials 119. Although not shown, the pair of registration fiducials 119 may be provided on one side of the arm. Furthermore, only one registration fiducial or three or more fiducials may be mounted relative to one or both of the front arms 121a, 121b in further embodiments. As further illustrated, the right rear arm 123a and the left rear arm 123b each comprise a single registration fiducial 119 although two or more registration fiducials may be provided in further embodiments.

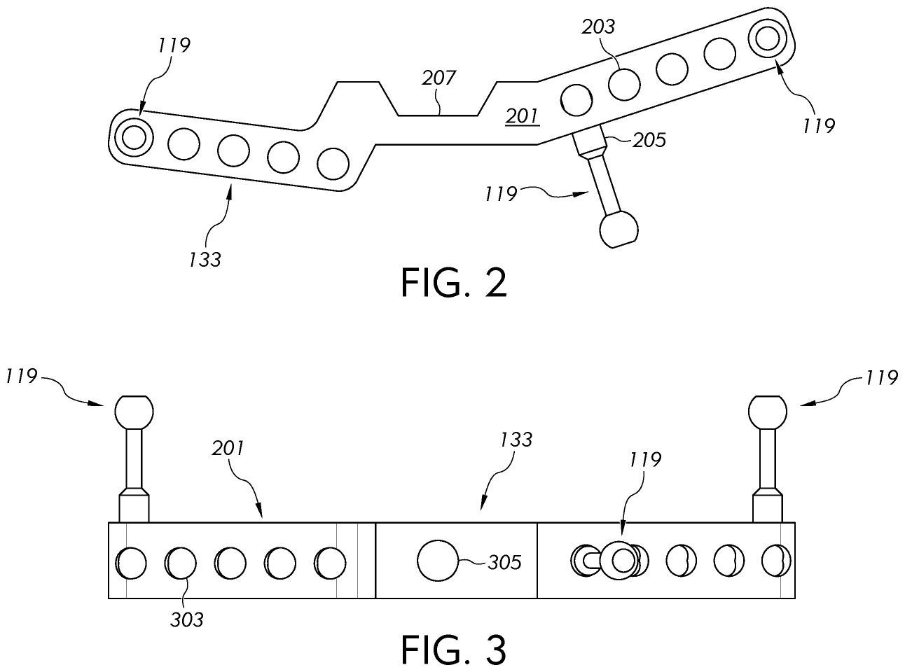

In some embodiments, the registration fiducials 119 can be mounted relative to the arm by being directly mounted to the arm. For instance, the registration fiducials may be adjustably mounted relative to the arm (e.g., in the direction of the axis 105). In some embodiments, as shown, the registration fiducials 119 can be mounted relative to the arms by a bracket. A wide range of brackets can be provided in accordance with aspects of the disclosure. For example, as shown in FIGS. 1-3, a front bracket 133 can be provided to mount one or more registration fiducials 119 to the right front arm 121a and/or the left front arm 121b. In the orientation shown in FIG. 2, the end surface 201 can comprise the top surface shown in the left front bracket mounted to the left front arm 121b. The same front bracket 133 could be used for the right front arm 121a if flipped upside down with the pair of registration fiducials 119 mounted on the opposite end surface to achieve the configuration shown in FIG. 1.

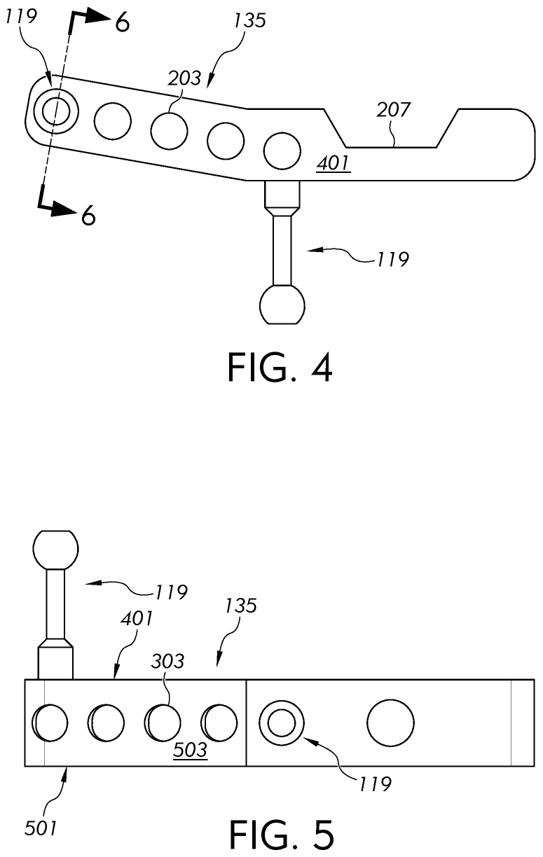

In further examples, as shown in FIGS. 1 and 4-5, a rear bracket 135 can be provided to mount one or more registration fiducials 119 to the right rear arm 123a and/or the left rear arm 123b. In the orientation shown in FIG. 4, the end surface 401 can comprise the top surface shown in the left rear bracket mounted to the left rear arm 123b. The same rear bracket 135 could be used for the right rear arm 123a if flipped upside down with the registration fiducial mounted on the opposite end surface to achieve the configuration shown in FIG. 1.

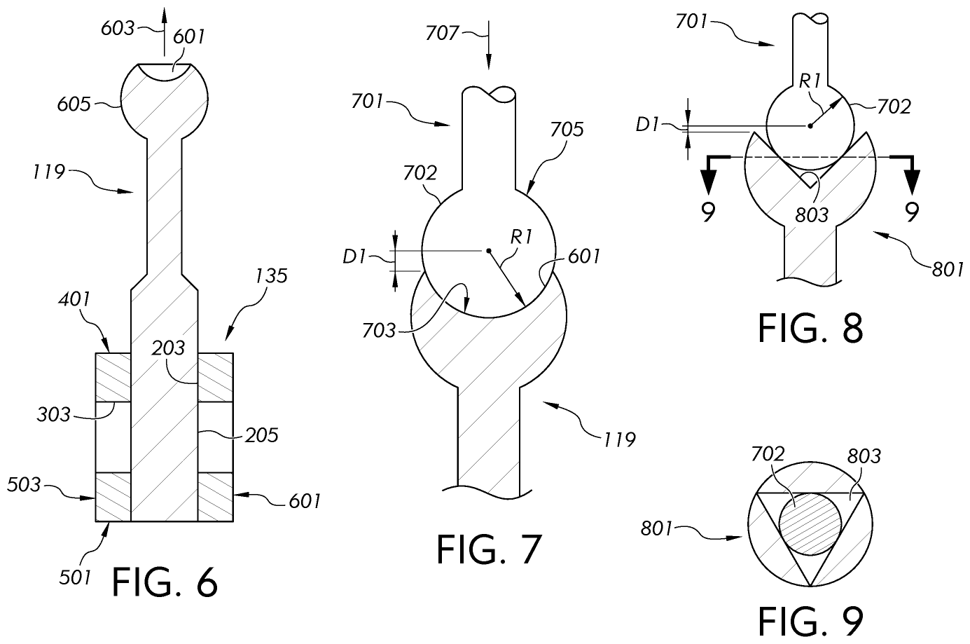

In some embodiments, the front bracket 133 and/or the rear bracket 135 can comprise one or more apertures configured to receive a shank 205 of the registration fiducial 119. For example, in some embodiments, as shown in FIGS. 2-5, the front bracket 133 and/or rear bracket 135 can comprise a plurality of apertures 203, 303. In some embodiments the shank can be threadingly received within a threaded aperture and then secured with a bolt or tack welded. In further embodiments, the shank may be press fit, welded or otherwise attached within the plurality of apertures. Providing a plurality of apertures can allow for customization of the stabilizing frame to allow different configurations by providing registration fiducials 119 at different locations on the bracket based on which aperture is selected to receive the registration fiducial. Furthermore, the apertures can be oriented in different directions to accommodate different approaches by the registration probe. For instance, as shown in FIG. 1, the first set of apertures 203 can each extend along an axis that is parallel to the axis 105 of the stabilizing frame 101. In further examples, the second set of apertures 303 can each extend along an axis in a direction that is perpendicular to a direction of the axis 105 of the stabilizing frame 101. In some embodiments, each apertures of the plurality of apertures 203, 303 can comprise through apertures extending from one surface to the opposite surface. For instance, as shown in FIG. 6, aperture 203 extends from the end surface 401 to the opposite end surface 501. Likewise, the aperture 303 extends from surface 503 to opposite surface 602.

The registration fiducial 119 can be adjustably mounted relative to the base 103. As such, adjustment of the location of the registration fiducial 119 relative to the base 103 can be achieved to customize the procedure and thereby optimize registration during the surgical procedure. In some embodiments, the registration fiducial 119 is adjustably attached (e.g., by an adjustable bracket) directly to the base. Adjustments while attached to the base can be provided in various directions such as a direction of the axis 105 of the stabilizing frame 101 or other directions. In further embodiments, as shown, the registration fiducial 119 is adjustably mounted relative to the arms 121a, 121b, 123a, 123b of the stabilizing frame 101 for selective adjustment in the direction of the axis 105 of the stabilizing frame 101. As shown, in some embodiments, the front bracket 133 and/or the rear bracket 135 can adjustably mount the registration fiducial 119 to one or more of the corresponding arms 121a, 121b, 123a, 123b. In some embodiments, the front bracket 133 and/or rear bracket 135 can be configured to adjustably mount the registration fiducial 119 for selective adjustment in a direction of the axis 105 of the stabilizing frame 101. For example, the front bracket 133 and/or rear bracket 135 can be adjustably keyed to a corresponding arm 121a, 121b, 123a, 123b for selective adjustable mounting in the direction of the axis 105. As shown in FIGS. 2 and 4, the front bracket 133 and the rear bracket 135 can each comprise a groove 207 configured to receive a correspondingly shaped tongue 137 (see FIG. 1) of the arm to key the bracket to the arm to define a linear sliding path. Furthermore, a fastener 139 extends through an aperture 305 (see FIG. 3), through the elongated slot 127 and into a follower 141. Once a desired elevation of the bracket relative to the arm is achieved, the fastener 139 can be tightened to lock the bracket (and registration fiducial 119) in position relative to the corresponding arm.

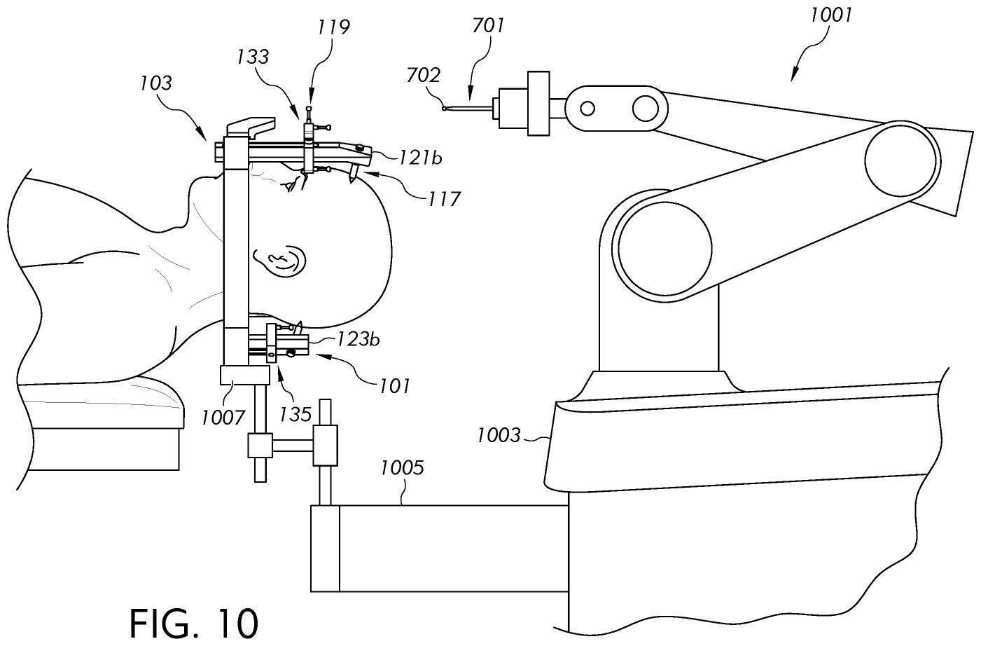

FIG. 6 illustrates a cross-sectional view of the registration fiducial 119 comprising a recess 601 configured to receive a tip 702 of a registration probe 701 of a robot 1001 (see FIG. 10). As shown in FIG. 6, the recess 601 comprises an opening that faces a direction 603 that can be oriented depending on the mounting arrangement relative to the base 103 of the stabilizing frame 101. For example, if the shank 205 of the registration fiducial 119 is mounted within one of the apertures 203, the direction 603 that the opening faces can comprise the direction of the axis 105 of the stabilizing frame 101. In another example, if the shank 205 of the registration fiducial 119 is mounted within one of the apertures 303, the direction 603 that the opening faces can comprise an outward direction perpendicular to the direction of the axis 105 and away from the head of the patient.

As shown in FIG. 6, in some embodiments, the distal tip 605 of the registration fiducial 119 that defines the recess 601 can comprise a bulbous portion. In some embodiments, as shown, the bulbous portion can comprise a partial outer spherical member. The partial outer spherical member can be easily located by imaging compared to other geometric shapes. As shown in FIG. 7, in some embodiments, the recess 601 of the registration fiducial 119 can be defined by a spherical inner surface segment 703 comprising a radius R1. Providing a spherical inner surface segment can be beneficial to provide surface symmetry that simplifies registration between the tip of the registration probe and the registration fiducial.

FIGS. 8-9 illustrate another embodiment of a registration fiducial 801 that can be similar or identical to the registration fiducial 119 discussed above unless otherwise specified. Rather than the spherical inner surface segment, the registration fiducial 801 comprises a pyramidal recess 803. FIG. 9 illustrates a triangular pyramidal recess although a square pyramidal recess or other pyramidal recess may be provided in further embodiments.

Methods will now be discussed for positioning a patient's head relative to a robot 1001 for a surgical procedure. As shown in FIG. 10, a patient's head and/or neck is positioned within the reception area 107 of the stabilizing frame 101 comprising the at least one registration fiducial 119. The stabilizing frame 101 can then be anchored to the skull of the head to removably fixedly attached the stabilizing frame 101 to the skull. For instance, a tool can be used to tighten the anchors 117 to embed the sharp tips of the anchors within the skull of the patient to fixedly attached the stabilizing frame 101 (that includes the at least one registration fiducial) to the skull. The method can further removably fixedly attach the stabilizing frame 101 to the robot 1001 to prevent the head from moving relative to a body 1003 of the robot 1001. For instance, a fixation arm device 1005 can connect the body 1003 to the base 103 of the stabilizing frame 101 with a bracket 1007. Once the fixation arm is attached, the head is immobilized and unable to move relative to the body 1003 of the robot 1001.

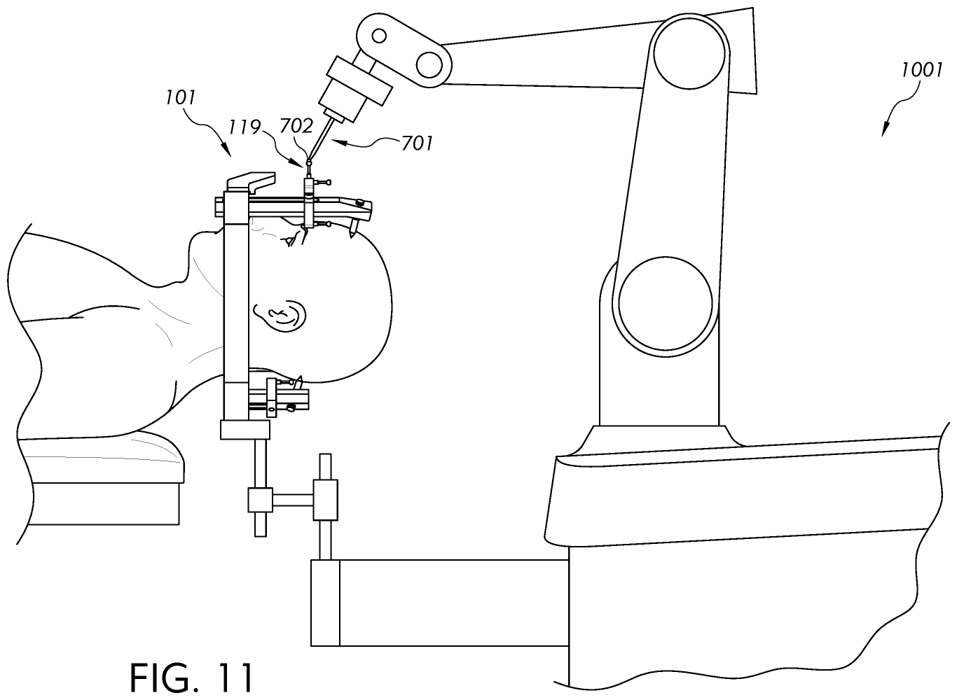

As shown in FIGS. 7 and 11, the registration probe 701 of the robot 1001 can be moved (e.g., manually) to insert the tip 702 of the probe into the recess 601 of the registration fiducial 119. For example, the tip 702 of the registration probe 701 can be inserted in direction 707 that is perpendicular to the face of the opening to the recess 601. In some embodiments, the direction 707 extends toward the skull wherein inserting the tip 702 of the registration probe 701 into the recess 601 of the registration fiducial 119 comprises moving the tip 702 in the direction 707 toward the skull of the patient.

As shown in FIG. 7, in some embodiments, the tip 702 of the registration probe 701 can comprise an outer spherical surface 705 and the recess 601 of the registration fiducial 119 can comprise the spherical inner surface segment 703. In some embodiments, as shown, the radius R1 of the outer spherical surface 705 of the tip 702 of the registration probe 701 is substantially equal to the radius R1 of the spherical inner surface segment 703 of the recess 601 of the registration fiducial 119. Providing matching radii can avoid rattling between the tip of the probe and the registration fiducial that may cause an error to occur within the programming of the robot.

As shown in FIG. 7, a depth of insertion of the tip 702 of the registration probe 701 into the recess 601 of the registration fiducial 119 is less than or equal to the radius R1 of the spherical inner surface segment 703 of the recess 601 of the registration fiducial 119 (e.g., by D1). In addition, or alternatively, a depth of insertion of the tip 702 of the registration probe 701 into the recess 601 of the registration fiducial 119 is less than or equal to the radius R1 of the outer spherical surface 705 of the tip 702 of the registration probe 701 (e.g., by D1). Limiting the depth of insertion of the tip of the probe as discussed herein can be beneficial to help prevent a collision between the tip and the registration fiducial that may cause an error to occur within the programming of the robot. Rather than colliding and interfering with one another, due to the limited insertion depth, the tip of the probe would simply ride out of the recess and thereby avoid registering as an error event with the robot.

Once the tip 702 of the registration probe 701 of the robot 1001 is finished being fully inserted into the recess 601 of the registration fiducial 119, the robot 1001 can store the position of the fiducial. The registration probe 701 can then be removed from the recess 601 of the registration fiducial 119 and moved to the next registration fiducial where the process is repleted until a sufficient number or all of the registration fiducial locations have been entered into the memory of the robot 1001. The robot 1001 can then use this information to align prior imaging of the brain with the locations obtained from the registration fiducials 119 to obtain the precise positioning of the brain orientation relative to the body 1003 of the robot 1001. The robot 1001 can then be used to conduct a wide range of surgical procedures on specific locations of the brain with precision.

In accordance with the disclosure, non-limiting aspects of the disclosure will now be described. Various combinations of the aspects can be provided in accordance with the disclosure.

Aspect 1. A stabilizing frame comprising a base extending at least 180 degrees about an axis of the stabilizing frame to define a reception area. The stabilizing frame further comprises a plurality of anchors mounted relative to the base, wherein the plurality of anchors are configured to be embedded within a skull of a patient. The stabilizing frame further comprises a registration fiducial mounted relative to the base, the registration fiducial comprising a recess configured to receive a distal tip of a registration probe of a robot.

Aspect 2. The stabilizing frame of Aspect 1, wherein the base extends 360 degrees about the axis to define the reception area.

Aspect 3. The stabilizing frame of Aspect 1, further comprising a plurality of arms mounted to the base, wherein each arm of the plurality of arms comprises a segment extending away from the base in a direction of the axis.

Aspect 4. The stabilizing frame of Aspect 3, wherein a corresponding anchor of the plurality of anchors is mounted to a distal end portion of each arm of the plurality of arms.

Aspect 5. The stabilizing frame of Aspect 3, wherein the segment of each arm of the plurality of arms is parallel to the axis.

Aspect 6. The stabilizing frame of Aspect 3, wherein each arm of the plurality of arms is adjustable relative to the base in the direction of the axis.

Aspect 7. The stabilizing frame of Aspect 3, wherein the registration fiducial is mounted relative a first arm of the plurality of arms.

Aspect 8. The stabilizing frame of Aspect 7, wherein the registration fiducial is adjustably mounted relative to the first arm the direction of the axis.

Aspect 9. The stabilizing frame of Aspect 1, wherein the registration fiducial is adjustably mounted relative to the base

Aspect 10. The stabilizing frame of Aspect 9, wherein the registration fiducial is adjustably mounted relative to the base in a direction of the axis.

Aspect 11. The stabilizing frame of Aspect 1, further comprising a bracket mounting the registration fiducial relative to the base.

Aspect 12. The stabilizing frame of Aspect 11, further comprising a plurality of arms mounted to the base, wherein the bracket mounts the registration fiducial to a first arm of the plurality of arms.

Aspect 13. The stabilizing frame of Aspect 12, wherein the bracket is adjustably mounted relative to the first arm in a direction of the axis.

Aspect 14. The stabilizing frame of Aspect 13, wherein the bracket is adjustably keyed to the first arm for selective adjustable mounting in the direction of the axis.

Aspect 15. The stabilizing frame of Aspect 11, wherein the bracket is adjustably mounted relative to the base in a direction of the axis.

Aspect 16. The stabilizing frame of Aspect 11, wherein the bracket comprises a plurality of apertures configured to selectively receive a shank of the registration fiducial.

Aspect 17. The stabilizing frame of Aspect 16, wherein the plurality of apertures each extend along an axis that is parallel to the axis of the stabilizing frame.

Aspect 18. The stabilizing frame of Aspect 16, wherein the plurality of apertures each extend along an axis in a direction that is perpendicular to a direction of the axis of the stabilizing frame.

Aspect 19. The stabilizing frame of Aspect 1, wherein the recess comprises an opening facing a direction of the axis.

Aspect 20. The stabilizing frame of Aspect 1, wherein the recess comprises an opening facing an outward direction that is perpendicular to a direction of the axis.

Aspect 21. The stabilizing frame of Aspect 1, wherein the recess of the registration fiducial comprises a spherical inner surface segment.

Aspect 22. The stabilizing frame of Aspect 1, wherein the recess of the registration fiducial comprises a pyramidal recess.

Aspect 23. A method of positioning a patient's head relative to a robot for a surgical procedure comprising positioning a patient's head and/or neck within a reception area of a stabilizing frame comprising a registration fiducial. The method further comprises anchoring the stabilizing frame to a skull of the head to removably fixedly attach the stabilizing frame to the skull. The method still further comprises removably fixedly attaching the stabilizing frame to the robot to prevent the head from moving relative to a body of the robot. The method still further comprises inserting a tip of a probe of the robot into a recess of the registration fiducial.

Aspect 24. The method of Aspect 23, wherein the tip of the probe comprises an outer spherical surface.

Aspect 25. The method of Aspect 24, wherein the recess of the registration fiducial comprises a spherical inner surface segment.

Aspect 26. The method of Aspect 25, wherein the radius of the outer spherical surface of the tip of the probe is substantially equal to the radius of the spherical inner surface segment of the recess of the registration fiducial.

Aspect 27. The method of Aspect 25, wherein a depth of insertion of the tip of the probe into the recess of the registration fiducial is less than or equal to a radius of the spherical inner surface segment of the recess of the registration fiducial.

Aspect 28. The method of Aspect 25, wherein a depth of insertion of the tip of the probe into the recess of the registration fiducial is less than or equal to a radius of the outer spherical surface of the tip of the probe.

Aspect 29. The method of Aspect 28, wherein a depth of insertion of the tip of the probe into the recess of the registration fiducial is less than or equal to a radius of the spherical inner surface segment of the recess of the registration fiducial.

Aspect 30. The method of Aspect 23, wherein inserting the tip of the probe into the recess of the registration fiducial comprises moving the tip in a direction toward the skull.

Aspect 31. The method of Aspect 23, further comprising attaching the registration fiducial to a portion of the stabilizing frame with a bracket.

Aspect 32. The method of Aspect 31, further comprising adjusting a position of the bracket relative to the portion of the stabilizing frame to adjust a position of the registration fiducial relative to the portion of the stabilizing frame.

Aspect 33. The method of Aspect 32, further comprising linearly sliding the bracket along a keyed path to linearly adjust the position of the bracket relative to the portion of the stabilizing frame to adjust the position of the registration fiducial relative to the portion of the stabilizing frame.

Although the disclosure has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the disclosure. In addition, while a particular feature of the disclosure may have been described above with respect to only one or more of several illustrated embodiments, such feature may be combined with one or more other features of the other embodiments, as may be desired and advantageous for any given or particular application.

What is claimed is:

1. A stabilizing frame comprising:
a base extending at least 180 degrees about an axis of the stabilizing frame to define a reception area;
a plurality of anchors mounted relative to the base, wherein the plurality of anchors are configured to be embedded within a skull of a patient; and
a registration fiducial mounted relative to the base, the registration fiducial comprising a recess configured to receive a distal tip of a registration probe of a robot.

2. The stabilizing frame of claim 1, further comprising a plurality of arms mounted to the base, wherein each arm of the plurality of arms comprises a segment extending away from the base in a direction of the axis.

3. The stabilizing frame of claim 2, wherein a corresponding anchor of the plurality of anchors is mounted to a distal end portion of each arm of the plurality of arms.

4. The stabilizing frame of claim 2, wherein each arm of the plurality of arms is adjustable relative to the base in the direction of the axis.

5. The stabilizing frame of claim 2, wherein the registration fiducial is mounted relative a first arm of the plurality of arms.

6. The stabilizing frame of claim 5, wherein the registration fiducial is adjustably mounted relative to the first arm the direction of the axis.

7. The stabilizing frame of claim 1, wherein the registration fiducial is adjustably mounted relative to the base.

8. The stabilizing frame of claim 7, wherein the registration fiducial is adjustably mounted relative to the base in a direction of the axis.

9. The stabilizing frame of claim 1, further comprising a bracket mounting the registration fiducial relative to the base.

10. The stabilizing frame of claim 9, further comprising a plurality of arms mounted to the base, wherein the bracket mounts the registration fiducial to a first arm of the plurality of arms.

11. The stabilizing frame of claim 10, wherein the bracket is adjustably mounted relative to the first arm in a direction of the axis.

12. The stabilizing frame of claim 11, wherein the bracket is adjustably keyed to the first arm for selective adjustable mounting in the direction of the axis.

13. The stabilizing frame of claim 9, wherein the bracket is adjustably mounted relative to the base in a direction of the axis.

14. The stabilizing frame of claim 9, wherein the bracket comprises a plurality of apertures configured to selectively receive a shank of the registration fiducial.

15. The stabilizing frame of claim 14, wherein the plurality of apertures each extend along an axis that is parallel to the axis of the stabilizing frame.

16. The stabilizing frame of claim 14, wherein the plurality of apertures each extend along an axis in a direction that is perpendicular to a direction of the axis of the stabilizing frame.

17. The stabilizing frame of claim 1, wherein the recess comprises an opening facing a direction of the axis.

18. The stabilizing frame of claim 1, wherein the recess comprises an opening facing an outward direction that is perpendicular to a direction of the axis.

19. The stabilizing frame of claim 1, wherein the recess of the registration fiducial comprises a spherical inner surface segment.

20. A method of positioning a patient's head relative to a robot for a surgical procedure comprising:
positioning a patient's head and/or neck within a reception area of a stabilizing frame comprising a registration fiducial;
anchoring the stabilizing frame to a skull of the head to removably fixedly attach the stabilizing frame to the skull;
removably fixedly attaching the stabilizing frame to the robot to prevent the head from moving relative to a body of the robot; and
inserting a tip of a probe of the robot into a recess of the registration fiducial.
